(19) United States
(12) Patent Application Publication
(10) Pub. No.: US 2024/0358314 A1
(43) Pub. Date: Oct. 31, 2024

(54) METHOD AND APPARATUS FOR DETERMINING THE LEVEL OF SEPSIS

(71) Applicant: CORESYS-HEALTH SL, Mataró (ES)

(72) Inventor: Mathieu JOSPIN, Mataró (ES)

(21) Appl. No.: 18/764,226

(22) Filed: Jul. 4, 2024

Related U.S. Application Data
(63) Continuation of application No. PCT/IB2022/062844, filed on Dec. 28, 2022.

(30) Foreign Application Priority Data
Jan. 5, 2022 (DK) PA202200009

(51) Int. Cl. A61B 5/00; A61B 5/0205; A61B 5/024; A61B 5/366

(52) U.S. Cl. CPC A61B 5/412; A61B 5/0205; A61B 5/366; A61B 5/4842; A61B 5/02405

(57) ABSTRACT

The continuous monitoring of electroencephalogram (EEG) and electrocardiogram (ECG) is crucial for detecting the degree of inflammation in a patient in a hospital setting, especially in the intensive care unit. In particular, the heart rate variability is known to correlate with the degree of inflammation. When the inflammation accelerates, this can lead to septic shock of the patient and subsequently multi-organ failure. Therefore, there is a need for a device that monitors the degree of sepsis of the patient. The present invention discloses a method and apparatus for monitoring the EEG and the ECG, and a method combining information from the EEG and the ECG which together with features extracted from the EEG and the ECG are the inputs to a prediction model such as an adaptive neuro fuzzy inference system the output of which is an index of sepsis.

### CROSS-REFERENCES TO RELATED APPLICATIONS

This application a bypass continuation of PCT application PCT/IB2022/062844 filed on Dec. 28, 2022 and published as WO2023/131856 which claims benefit of priority to Danish application PA202200009 filed on Jan. 5, 2022.

### FIELD OF THE INVENTION

The present invention relates to a method and apparatus for determining the level of sepsis in a subject.

### BACKGROUND OF THE INVENTION

During a hospital stay, the risk of getting an infection sensibly increases due to the concentrated exposure to germs it implies, the need for surgery or the use of invasive devices such as a central line, a urinary catheter or a mechanical ventilator, or the presence of pressure injuries due to reduced mobility. In hospital environments, many people are also more susceptible to infection than general population because of chronic illness, age, or other risk factors.

In some cases, the reaction of the immune system triggered by infection gets out of control and gives rise to sepsis, a life-threatening condition. Sepsis is defined as infection with evidence of a systemic inflammatory process, with at least two of the following symptoms: 1) increased or decreased temperature or leucocyte count; 2) tachycardia; 3) tachypnea. Septic shock, a type of sepsis with hypotension persisting after resuscitation with intravenous fluids, may happen as a consequence of the worsening of the inflammation and acute failure of multiple organs, including the lungs, kidneys, and liver, may occur.

An early diagnostic followed by treatment with antibiotics and fluids may help reverse this adverse situation. Therefore, there is a need for a device that monitors the degree of sepsis of the patient and can give an early notice in case of a deteriorating state of the patient.

The U.S. Pat. No. 7,941,199 B2 discloses a non-invasive sepsis monitoring system which may include the recording of ECG and EEG signals, but does not use HRV, HRnV nor a prediction model such as ANFIS for example. It also does not derive features from the simultaneous comparison of features from the EEG with features from the ECG. Hence the present invention is significantly different.

The US patent US 2006/0155176 A1 discloses a method for continuous monitoring of patients to detect the potential onset of sepsis which includes the recording of an ECG and use of HRV, but it does not make use of HRnV, EEG, nor does it derive features from the simultaneous comparison of features from the EEG with features from the ECG. It also does not refer to the use of a prediction model such as ANFIS for example. Hence the present invention is substantially different.

The US patent US 2016/0374581 A1 discloses an apparatus for estimating systemic inflammation including EEG and ECG processing. In the US patent US 2016/0374581 A1, the EEG is recorded solely from frontal electrodes, whereas in the present invention at least one electrode is positioned above one of the cars of the patient, where the best signal-to-noise ratio of the EEG from insular cortex can be achieved. In the US patent US 2016/0374581 A1, the features extracted from the sole EEG are restricted to an index of the hypnotic effect, whereas in the present invention a much broader range of EEG features are considered comprising time domain features related to the detection and quantification of specific patterns such as the burst suppression pattern and frequency domain features such as the energy content in any EEG frequency bands and the energy ratios across pairs of any EEG frequency bands. The US patent US 2016/0374581 A1 refers to the use of HRV solely, whereas the present invention makes use of both HRV and HRnV. Finally, to represent the joint information carried by the EEG and the ECG, the US patent US 2016/0374581 A1 uses transfer entropy whereas the present invention uses cross-correlation and mutual information. Hence the present invention is significantly different.

The U.S. Pat. No. 10,299,689 B2 discloses a system and method with a different purpose than the present invention, it provides an assessment of risk of a cardiac event for triage purpose only, while the present invention consists in continuous monitoring to determine the level of sepsis. The U.S. Pat. No. 10,299,689 B2 includes the use of ECG and HRV parameters, but it does not make use of HRnV, EEG, nor does it derive features from the simultaneous comparison of features from the EEG with features from the ECG. Hence the present invention is substantially different.

### SUMMARY OF THE INVENTION

The present invention provides a method for passively and non-invasively determining the level of sepsis on critically ill patients attended in hospital environments such as intensive care units (ICUs) or emergency departments (EDs), comprising the steps of: a) recording EEG (3) and ECG (4) signals from a patient; b) extracting features from the EEG; c) extracting features from the ECG; d) computing features based on the simultaneous comparison of features from the EEG with features from the ECG; e) defining an index of sepsis as a function of the features extracted from the EEG, the features extracted from the ECG and the features derived from the simultaneous comparison of EEG and ECG features.

The features considered to be extracted from the EEG are time domain features such as a quantification of the burst suppression pattern and frequency domain features such as the energy content in EEG frequency bands and the energy ratios across pairs of EEG frequency bands.

The features considered to be extracted from the ECG are the heart rate (HR) as well as time domain, frequency domain and nonlinear features provided by the analysis of the heart rate variability (HRV) and heart rate n-variability (HRnV).

The features considered for the simultaneous comparison of features from the EEG with features of the ECG are derived from the cross-correlation and mutual information functions between the energy content and energy ratios in EEG, HRV and HRnV frequency bands.

### BRIEF DESCRIPTION OF THE FIGURES

These and other features, aspects, and advantages of the present invention will become better understood with regard to the following description, appended claims and accompanying drawings, where:

FIG. 1 details the features derived from the electrical recording on the scalp and the chest of the patient and their combination in order to achieve a sepsis index.

FIG. 2 presents a possible configuration of the electrodes used to record the EEG signals.

FIG. 3 introduces the heart rate n-variability framework by illustrating the construction of the related series of intervals, referred to as RRI_{n,m}, for several combinations of n and m, n being the number of considered consecutive individual interbeat intervals and m the shift expressed in individual interbeat intervals between consecutive new intervals.

FIG. 4 presents a possible display for the signals and indices provided by a microprocessor implementing the methods described in the present invention.

### DETAILED DESCRIPTION OF THE EMBODIMENTS

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide method and apparatus for determining the level of sepsis by combination of parameters extracted from an electroencephalogram and an electrocardiogram.

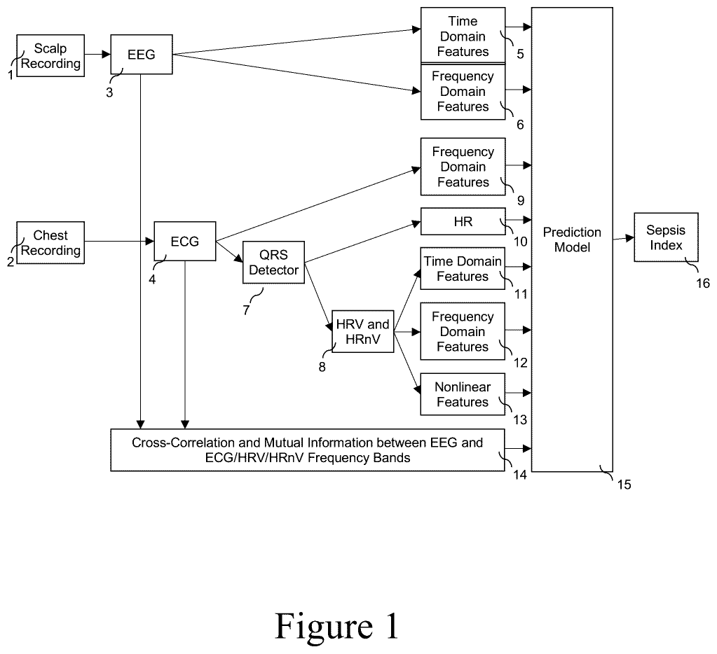

FIG. 1 details the features derived from the electrical recording on the scalp and the chest of the patient and their combination in order to achieve a sepsis index.

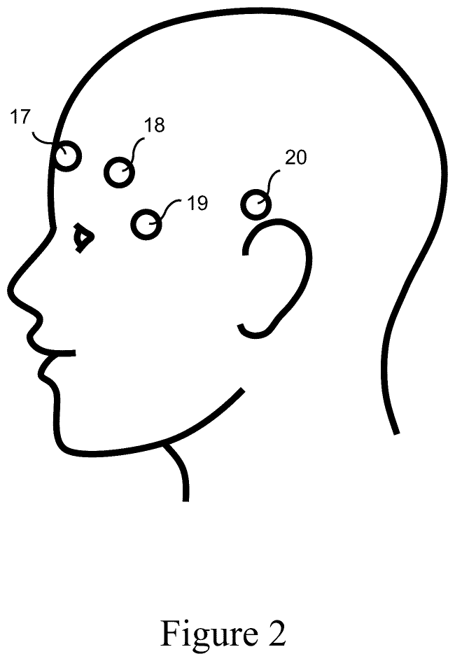

In the embodiment, one or more EEG (3) signals are recorded from the patient's scalp (1). As shown in FIG. 2, at least 3 electrodes (17, 18 and 19) are positioned on the forehead and at least 1 electrode is located above one of the ears (20), where the best signal-to-noise ratio of the EEG from insular cortex can be achieved.

Time domain features (5) are extracted from the EEG signals to detect and quantify specific patterns such as the burst suppression pattern. An FFT is also applied to these EEG signals enabling the calculation of frequency domain features such as the energy content in EEG frequency bands and the energy ratios across pairs of EEG frequency bands (6).

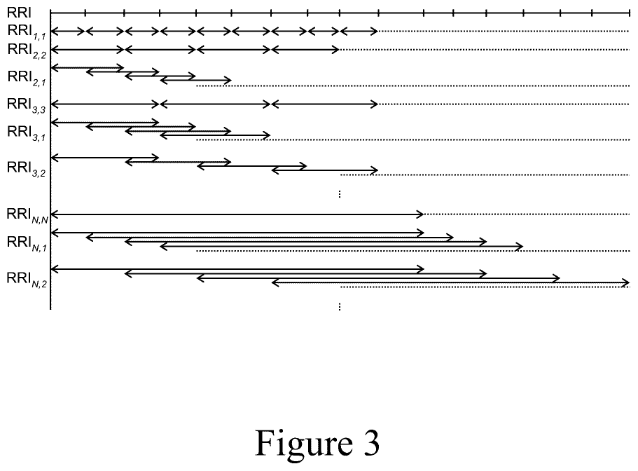

In the embodiment, one or more ECG (4) signals are recorded using 2 or more electrodes positioned on the patient's chest (2). An FFT is applied to these ECG signals enabling the calculation of frequency domain features (9) such as the energy content in ECG frequency bands and the energy ratios across pairs of ECG frequency bands. The detection of the location of the QRS complexes (7) is achieved by applying the Pan-Tompkins algorithm or a variation of it to the ECG, which is used to measure the intervals between consecutive heartbeats, or R-R intervals, from which the heart rate (HR) (10) is then derived. The location of the QRS complexes (7) is also used to build R-R interval series (RRI), which serve as the basis of both heart rate variability (HRV) and heart rate n-variability (HRnV) (8). HRnV was first introduced in Heart Rate n-Variability (HRnV): A Novel Representation of Beat-to-Beat Variation in Electrocardiogram, by Nan Liu, Dagang Guo, Zhi Xiong Koh, Andrew Fu Wah Ho and Marcus Eng Hock Ong, bioRxiv 2018:449504. Instead of exploring beat-to-beat variations of the ECG on the basis of the series of the consecutive individual RR intervals (RRI) as HRV does, HRnV builds series of intervals resulting from the sum of multiple consecutive RR intervals, with or without overlapping. The series of new intervals provided by HRnV may be referred to as RRI_{n,m}, where n represents the number of considered consecutive individual RR intervals and m the shift expressed in individual RR intervals between consecutive new intervals. The valid ranges for n and m are 1≤n≤N and 1≤m≤n where Nis chosen so that N<<Nₜₒₜ, Nₜₒₜ being the total number of RR intervals available for processing, in order to provide sufficient data points for analysis. The case m=n corresponds to series of non-overlapping intervals. The remaining cases with m such as 1≤m<n correspond to series with overlapping, the length of the overlap expressed in RR intervals being given by n-m. FIG. 3 illustrates the construction of RRI_{n,m} for several combinations of n and m. It is of note that RRI_{1,1} corresponds to the original RRI series used in classic HRV.

Time domain features (11) are extracted from the RRI and RRI_{n,m} series, being for example: 1) the root mean square differences between successive intervals (RMSSD); 2) the standard deviation of the differences between successive intervals (SDSD); 3) the percentage of successive intervals differing by more than 50 ms (pNN50); 4) the standard deviation of the intervals, typically computed over a 24-hour period (SDNN); 5) the standard deviation of the average intervals computed over short periods, typically 5 minutes (SDANN).

Frequency domain features (12) are extracted from the RRI and RRI_{n,m} series, being for example: 1) the power below 0.04 Hz, the very low frequency range (VLF); 2) the power between 0.04 Hz and 0.15 Hz, the low frequency range (LF); 3) the power between 0.15 Hz and 0.4 Hz, the high frequency range (HF); 4) the normalized power in the low frequency range (nLF), defined as nLF=LF/(LF+HF)*100; 5) the normalized power in the high frequency range (nHF), defined as nHF=HF/(LF+HF)*100; 6) the ratio of the power in the low frequency range and the power in the high frequency range (LF/HF).

Nonlinear features (13) are extracted from the RRI and RRI_{n,m} series, being for example: 1) the approximate entropy (ApEn); 2) the sample entropy (SampEn); 3) the coefficients α₁ and α₂ provided by the detrended fluctuation analysis (DFA).

The combined information of features from the EEG with features of the ECG is derived from the cross-correlation and mutual information functions between the energy content and energy ratios in EEG frequency bands and the energy content and energy ratios in ECG, HRV and HRnV frequency bands (14).

The features extracted from the EEG, the features extracted from the ECG and the features derived from the simultaneous comparison of EEG and ECG features are used as inputs to the prediction model (15) which can be either a linear regression, a logistic regression, a fuzzy logic classifier, a neural network, a hybrid between a fuzzy logic system and a neural network such as an adaptive neuro fuzzy inference system (ANFIS), or any other prediction model. The output of the prediction model is the sepsis index (16).

### Graphical Presentation

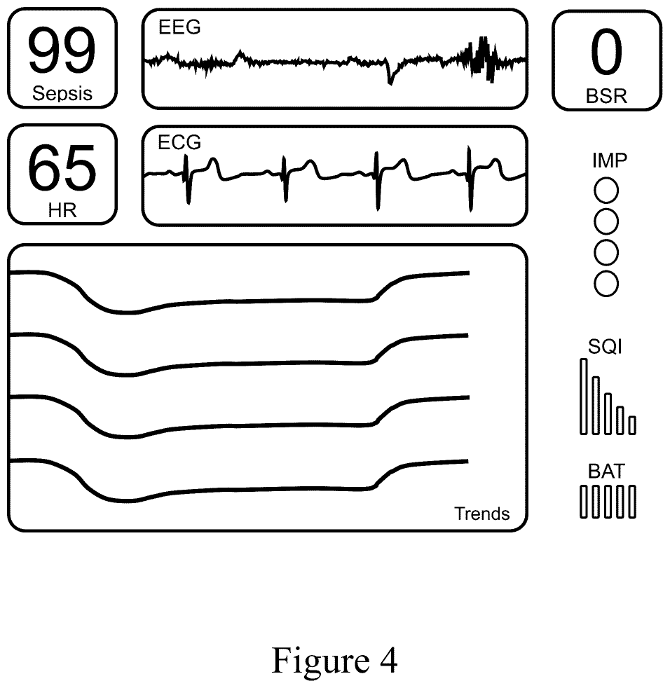

The methods presented in the present invention are implemented into a microprocessor where the output to a display (FIG. 4), among others, may be any of the following: 1) one or several EEG signals; 2) one or several ECG signals; 3) the value of the level of sepsis; 4) the value of the heart rate (HR); 5) the value of the burst suppression ratio (BSR); 6) the value of the impedance of the electrodes (IMP); 7) the value of a signal quality index (SQI); 8) the value of the level of the battery (BAT); 9) the trend of any of the calculated indices over time.

1. A method for determining the level of sepsis by combination of parameters extracted from an electroencephalogram (3) and an electrocardiogram (4) comprising the following steps:
a. measuring the electroencephalogram (3);
b. measuring the electrocardiogram (4);
c. detecting the location of the QRS complexes (7) in the electrocardiogram (4);
d. building the interbeat interval series used for the calculation of the heart rate variability and the heart rate n-variability (8);
e. calculating the time domain features from the electroencephalogram (5);
f. calculating the frequency domain features from the electroencephalogram (6);
g. calculating the frequency domain features from the electrocardiogram (9,28);
h. calculating the heart rate (10) from the location of the QRS complexes;
i. calculating time domain features from the heart rate variability and heart rate n-variability (11);
j. calculating frequency domain features from the heart rate variability and heart rate n-variability (12);
k. calculating nonlinear features from the heart rate variability and heart rate n-variability (13);
l. calculating the cross-correlation and the mutual information (12) between the electroencephalogram (3) and the electrocardiogram (4), the heart rate variability and the heart rate n-variability (8);
m. using a prediction model (15) to combine at least four parameters derived from the electroencephalogram (3) and the electrocardiogram (4) into a final index of sepsis (16).

2. The method according to claim 1, wherein step d is characterized by the construction of series of the consecutive individual interbeat intervals in the case heart rate variability and the construction of series of intervals resulting from the sum of multiple consecutive interbeat intervals, with or without overlapping, in the case of heart rate n-variability.

3. The method according to claim 1, wherein step g is characterized by the extraction of frequency domain features from the electrocardiogram (4) such as the energy content in frequency bands of the electrocardiogram and the energy ratios across pairs of frequency bands of the electrocardiogram.

4. The method according to claim 1, wherein step i is characterized by the extraction of time domain features from the series of intervals considered in both heart rate variability and heart rate n-variability such as the root mean square differences between successive intervals (RMSSD), the standard deviation of the differences between successive intervals (SDSD), the percentage of successive intervals differing by more than 50 ms (pNN50), the standard deviation of the intervals typically computed over a 24-hour period (SDNN), or the standard deviation of the average intervals computed over short periods, typically 5 minutes (SDANN).

5. The method according to claim 1, wherein step j is characterized by the extraction of frequency domain features from the series of intervals considered in both heart rate variability and heart rate n-variability

6. The method according to claim 1, wherein step k is characterized by the extraction of nonlinear features from the series of intervals considered in both heart rate variability and heart rate n-variability such as the approximate entropy (ApEn), the sample entropy (SampEn) or the coefficients α₁ and α₂ provided by the detrended fluctuation analysis (DFA).

7. The method according to claim 1, wherein step 1 is characterized by calculating features derived from the cross-correlation and mutual information functions between the energy content and energy ratios of the electroencephalogram (3) and the energy content and energy ratios of the electrocardiogram (4) and of the series of intervals considered in both heart rate variability and heart rate n-variability.

8. The method according to claim 1, wherein step m is characterized by the use of a prediction model which can be either a linear regression, a logistic regression, a fuzzy logic classifier, a neural network, a hybrid between a fuzzy logic system and a neural network such as an adaptive neuro fuzzy inference system, or any other prediction model.

9. The method according to claim 1, implemented into a microprocessor where the output to a display, among others, may be any of the following: one or several EEG signals, one or several ECG signals, the value of the level of sepsis, the value of the heart rate (HR), the value of the burst suppression ratio (BSR), the value of the impedance of the electrodes (IMP), the value of a signal quality index (SQI), the value of the level of the battery (BAT) or the trend of any of the calculated indices over time.

10. An apparatus for determining a level of sepsis by combination of parameters extracted from an electroencephalogram (3) and an electrocardiogram (4) comprising:
a. a sensor for measuring the electroencephalogram (3);
b. a sensor for measuring the electrocardiogram (4);
c. a microprocessor configured to:
i. detect the location of the QRS complexes (7) in the electrocardiogram (4);
ii. build the interbeat interval series used for the calculation of the heart rate variability and the heart rate n-variability (8);
iii. calculate the time domain features from the electroencephalogram (5);
iv. calculate the frequency domain features from the electroencephalogram (6);
v. calculate the frequency domain features from the electrocardiogram (9,28);
vi. calculate the heart rate (10) from the location of the QRS complexes;
vii. calculate time domain features from the heart rate variability and heart rate n-variability (11);
viii. calculate frequency domain features from the heart rate variability and heart rate n-variability (12);
ix. calculate nonlinear features from the heart rate variability and heart rate n-variability (13);
x. calculate the cross-correlation and the mutual information (12) between the electroencephalogram (3) and the electrocardiogram (4), the heart rate variability and the heart rate n-variability (8);
xi. use a prediction model (15) to combine at least four parameters derived from the electroencephalogram (3) and the electrocardiogram (4) into a final index of sepsis (16).

11. The apparatus according to claim 10, wherein step a is characterized by a sensor consisting of 3 or more electrodes positioned on the forehead (17, 18 and 19) and 1 or more electrodes above the ear on one or both sides of the subject recording electroencephalogram from the insular cortex (20).

12. The apparatus according to claim 10, wherein said configured to build the interbeat interval series further comprises constructing series of the consecutive individual interbeat intervals in the case heart rate variability and the construction of series of intervals resulting from the sum of multiple consecutive interbeat intervals, with or without overlapping, in the case of heart rate n-variability.

13. The apparatus according to claim 10, wherein said configured to calculate the frequency domain features from the electroencephalogram further comprises extracting frequency domain features from the electroencephalogram (3) such as the energy content in frequency bands of the electroencephalogram and the energy ratios across pairs of frequency bands of the electroencephalogram.

14. The apparatus according to claim 10, wherein said configured to calculate the frequency domain features from the electrocardiogram further comprises extracting frequency domain features from the electrocardiogram (4) such as the energy content in frequency bands of the electrocardiogram and the energy ratios across pairs of frequency bands of the electrocardiogram.

15. The apparatus according to claim 10, wherein said configured to calculate time domain features from the heart rate variability and heart rate n-variability further comprises extracting time domain features from the series of intervals considered in both heart rate variability and heart rate n-variability such as the root mean square differences between successive intervals (RMSSD), the standard deviation of the differences between successive intervals (SDSD), the percentage of successive intervals differing by more than 50 ms (pNN50), the standard deviation of the intervals typically computed over a 24-hour period (SDNN), or the standard deviation of the average intervals computed over short periods, typically 5 minutes (SDANN).

16. The apparatus according to claim 10, wherein said configured to calculate frequency domain features from the heart rate variability and heart rate n-variability further comprises extracting frequency domain features from the series of intervals considered in both heart rate variability and heart rate n-variability such as the power below 0.04 Hz corresponding to the very low frequency range (VLF), the power between 0.04 Hz and 0.15 Hz corresponding to the low frequency range (LF), the power between 0.15 Hz and 0.4 Hz corresponding to the high frequency range (HF), the normalized power in the low frequency range (nLF) defined as nLF=LF/(LF+HF)*100, the normalized power in the high frequency range (nHF) defined as nHF=HF/(LF+HF)*100, or the ratio of the power in the low frequency range and the power in the high frequency range (LF/HF).

17. The apparatus according to claim 10, wherein said configured to calculate nonlinear features from the heart rate variability and heart rate n-variability further comprises extracting nonlinear features from the series of intervals considered in both heart rate variability and heart rate n-variability such as the approximate entropy (ApEn), the sample entropy (SampEn) or the coefficients α₁ and α₂ provided by the detrended fluctuation analysis (DFA).

18. The apparatus according to claim 10, wherein said configured to calculate the cross-correlation and the mutual information between the electroencephalogram and the electrocardiogram, the heart rate variability and the heart rate n-variability further comprises calculating features derived from the cross-correlation and mutual information functions between the energy content and energy ratios of the electroencephalogram (3) and the energy content and energy ratios of the electrocardiogram (4) and of the series of intervals considered in both heart rate variability and heart rate n-variability.

19. The apparatus according to claim 10, wherein said configured to use a prediction model further comprises a prediction model which can be either a linear regression, a logistic regression, a fuzzy logic classifier, a neural network, a hybrid between a fuzzy logic system and a neural network such as an adaptive neuro fuzzy inference system, or any other prediction model.

20. The apparatus according to claim 11 comprising a microprocessor configured to present an output to a display, among others, which may be any of the following: one or several EEG signals, one or several ECG signals, the value of the level of sepsis, the value of the heart rate (HR), the value of the burst suppression ratio (BSR), the value of the impedance of the electrodes (IMP), the value of a signal quality index (SQI), the value of the level of the battery (BAT) or the trend of any of the calculated indices over time.
